(19) United States
(12) Patent Application Publication
(10) Pub. No.: US 2009/0198272 A1
(43) Pub. Date: Aug. 6, 2009

(54) METHOD AND APPARATUS FOR ARTICULATING THE WRIST OF A LAPAROSCOPIC GRASPING INSTRUMENT

(76) Inventors: Lawrence Kerver, Los Gatos, CA (US); Brian Tang, Fremont, CA (US); Friedrich Ho, Mountain View, CA (US); Ben Nordell, San Mateo, CA (US)

Correspondence Address: GLENN PATENT GROUP, 3475 EDISON WAY, SUITE L, MENLO PARK, CA, 94025, US

(21) Appl. No.: 12/027,231

(22) Filed: Feb. 6, 2008

(51) Int. Cl. A61B 17/00; A61B 17/32

(52) U.S. Cl. 606/205; 606/206

(57) ABSTRACT

A medical instrument has a set of opposing jaws that can be articulated, both left and right, from centerline. The instrument has a proper bend radius and support for the jaw actuation member and cutter driving member. The bendable support for the drive members comprises tightly wound coil springs. Another embodiment of the invention controls the degree of articulation at the handle of the laparoscopic instrument. A further embodiment of the invention incorporates a locking mechanism to prevent motion of the wrist while the user performs other operations on the device. The locking mechanism also includes an indexing feature with which the user can index and choose the necessary amount of angle between preset angles.

### BACKGROUND OF THE INVENTION

1. Technical Field

The invention relates to medical devices for use during laparoscopic procedures. More particularly, the invention relates to a method and apparatus for articulating the wrist of a laparoscopic grasping instrument.

2. Description of the Prior Art

Laparoscopic surgery, also called minimally invasive surgery (MIS), band aid surgery, keyhole surgery, or pinhole surgery is a modern surgical technique in which operations in the abdomen are performed through small incisions, usually 0.5-1.5 cm, as compared to larger incisions needed in traditional surgical procedures. Laparoscopic surgery includes operations within the abdominal or pelvic cavities, whereas keyhole surgery performed on the thoracic or chest cavity is called thoracoscopic surgery. Laparoscopic and thoracoscopic surgery belong to the broader field of endoscopy.

The key element in laparoscopic surgery is the use of a laparoscope: a telescopic rod lens system, that is usually connected to a video camera (single chip or three chip). Also attached is a fiber optic cable system connected to a cold light source (halogen or xenon), to illuminate the operative field, inserted through a 5 mm or 10 mm cannula to view the operative field. The abdomen is usually insufflated with carbon dioxide gas to create a working and viewing space. The abdomen is essentially blown up like a balloon (insufflated), elevating the abdominal wall above the internal organs like a dome. The gas used is CO2, as it is common to the human body and can be removed by the respiratory system if it absorbs through tissue. It is also non-flammable, which is important due to the fact that electrosurgical devices are commonly used in laparoscopic procedures.

Surgery is performed during a laparoscopic procedure with any of various tools that are typically arranged on one end of a long shaft and that are operable by manipulation of a handle or other actuator positioned at the other end of the shaft.

One area of laparoscopic surgery that is currently the subject of interest is that of electrocauterization. Electrocauterization, also called electric surgery or electrosurgery, is the process of destroying tissue with electricity and is widely used in modern surgery. The procedure is frequently used to stop bleeding of small vessels, larger vessels being ligated, or for cutting through soft tissue, i.e. abdominal fat in a laparotomy or breast tissue in a mastectomy.

One problem with state of the art electrocauterization devices for use during a laparoscopic procedure is the limited range of motion provided by the jaws of such device, and the difficulty encountered by a surgeon in positioning such device, and in operating such device through a range of motion, during a laparoscopic procedure.

### SUMMARY OF THE INVENTION

The invention provides a method and apparatus for articulating the wrist of a laparoscopic grasping instrument. The presently preferred medical instrument has a set of opposing jaws that can be articulated, both left and right, from centerline. The invention also provides a proper bend radius and support for the jaw actuation member and cutter driving member. The bendable support for the drive members in the presently preferred embodiment of the invention comprises tightly wound coil springs.

Another embodiment of the invention incorporates a method to control the degree of articulation at the handle of the laparoscopic instrument. A further embodiment of the invention incorporates a locking mechanism to prevent motion of the wrist while the user performs other operations on the device. The locking mechanism also includes an indexing feature with which the user can index and choose the necessary amount of angle between preset angles.

### BRIEF DESCRIPTION OF THE DRAWINGS

FIG. 1 is a perspective diagram showing the wrist of a laparoscopic grasping instrument according to the invention;

FIG. 2 is a plan view showing a wrist of a laparoscopic grasping instrument according to the invention;

FIG. 3 is a schematic view showing a top cutaway of a wrist articulation control mechanism according to the invention;

FIG. 4 is a perspective schematic view showing a laparoscopic grasping instrument according to the invention;

FIG. 5 is another perspective view of a laparoscopic grasping instrument according to the invention;

FIG. 6 is a perspective schematic view of an indexing mechanism for a laparoscopic grasping instrument according to the invention;

FIG. 7 is a perspective schematic view of a detent mechanism for a laparoscopic grasping instrument according to the invention;

FIG. 8 is a perspective schematic view of a detent and indexing mechanism for a laparoscopic grasping instrument according to the invention;

FIG. 9 is a plan schematic view of a step ball detent mechanism for a laparoscopic grasping instrument according to the invention;

FIG. 10 is a perspective schematic view of the step ball detent mechanism for a laparoscopic grasping instrument according to the invention;

FIG. 11 is a second perspective schematic view of the step ball detent mechanism for a laparoscopic grasping instrument according to the invention;

FIG. 12 is a perspective schematic view of a push lock mechanism for an articulation control in a laparoscopic grasping instrument according to the invention;

FIG. 13 is a phantom perspective schematic view of the push lock mechanism for an articulation control mechanism in a laparoscopic grasping instrument according to the invention;

FIG. 14 is a perspective schematic view of a grab knob for the push lock mechanism in a articulation control for a laparoscopic grasping instrument according to the invention;

FIG. 15 is a perspective, partially cutaway view of a laparoscopic device, showing a drive member according to the invention; and

FIG. 16 is a perspective view of a drive assembly for a laparoscopic device blade according to the invention.

### DETAILED DESCRIPTION OF THE INVENTION

The invention provides a method and apparatus for articulating the wrist of a laparoscopic grasping instrument. The presently preferred medical instrument has a set of opposing jaws that can be articulated, e.g. 45 degrees or otherwise as desired, both left and right, from centerline. The invention also provides a proper bend radius and support for the jaw actuation member and cutter driving member. The bendable support for the drive members in the presently preferred embodiment of the invention comprises tightly wound coil springs.

Another embodiment of the invention incorporates a method to control the degree of articulation at the handle of the laparoscopic instrument. A further embodiment of the invention incorporates a locking mechanism to prevent motion of the wrist while the user performs other operations on the device. The locking mechanism also includes an indexing feature with which the user can index and choose the necessary amount of angle between preset angles.

The presently preferred embodiment of the invention comprises a medical instrument, preferably for performing a laparoscopic procedure, which comprises a set of pivotal vertebra that are connected to each other by pins or by a snap fit. Each vertebra is adapted to pivot in relation to a device shaft and jaw set, thus allowing left and right articulation. The degree of articulation is controlled by wires or cables that run down both sides of a device wrist. The wires are then routed down the shaft and connected in tension to a control mechanism at a device handle. The cables or wires are used to transfer the forces from the handle to the wrist.

The vertebra form the proper bend radius to allow for a force transfer member, such as a wire, to pass through the wrist without kinking the wire. Furthermore, in one embodiment a tightly wound coil spring is housed within the wrist joints to route said wire. The tightly wound coil spring provides additional support to the wire, such that when the wire is moved from a proximal to a distal direction, it does not buckle or kink.

The control mechanism at the handle consists of a rotating assembly that receives the force transfer members from the wrist. The rotating assembly is pivotally mounted at the handle, and the shape of the control mechanism allows for concentric rotation about the pivot so that the length-wise motion of the wires or cables along the shaft can be controlled, based upon the distance from the pivot to the attachment point of said wires or cables. The angle of articulation is controlled by the distance that the force transfer member moves, which is predetermined by the wrist geometry.

There are several embodiments that comprise a locking and indexing feature of the invention:

In a first embodiment, a spring steel is formed into a geometry that deflects when a force is applied, as with a leaf spring. The leaf spring is housed within a circular carrier, with only the deflectable portion of the spring accessible and protruding from a circular carrier. A rotating member with a circular portion removed from its pivot area fits over the circular carrier. A tooth pattern is also removed from along the inner diameter of the circular portion of the rotating member. The rotating member includes arms extending from its center body to which the cable or wires are attached. The leaf-like spring protrudes into the indentations created by the tooth pattern. The angle of articulation is controlled by predetermining the distances between the teeth and the distance from the attachment point of the cable or wires to the pivot point.

In a second embodiment, a spring plunger is mounted within the circular carrier. The spring plunger mates with the indents created by the tooth pattern.

In a third embodiment, the rotating member described above does not have arms extending from its center body. A wing is mounted on top of the rotating member. The wing is then manipulated to control the rotation around the circular carrier.

In a fourth embodiment, a living plastic hinge is mounted near the handle. The living plastic hinge uses a V-shape that fits within a slot of an external housing that surrounds the living hinge. The tip of the V-shape protrudes from each slot. There are a series of slots along the length of the external housing. The housing engages with the cable and wires that control articulation of the wrist. The user can adjust and lock the wrist articulation by first pressing down on the living hinge to disengage the current locked position, then moving the external housing from a proximal to a distal position or vice versa, which then locks by re-engaging with the living hinge at any various predetermined distances set by the slots. These distances determine the angle at which the wrist is articulated.

In a fifth embodiment, the rotating mechanism described above rotates freely around the pivot. When the user has determined the angle of articulation, a button mounted on top of the pivot is depressed, which locks the wrist angle and the rotating mechanism, thus preventing any further movement of both the rotating mechanism and wrist. This can be accomplished using a wedge-like design that is anchored within the pivot pin, which in this embodiment is a tube. A minimum of a single slot is designed into the pivot pin. When the button is depressed, the inherent spring properties of the button flare from the slot. The flaring material uses friction to prevent movement of the rotating mechanism. The button itself remains in place due to a wedge design at the top.

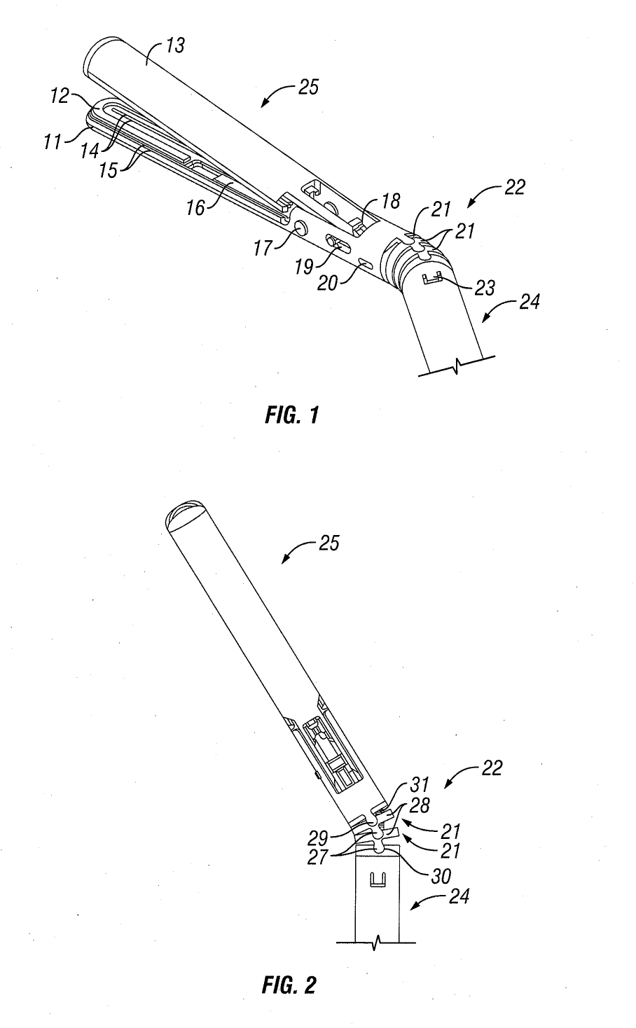

Further to the foregoing discussion, a more detailed explanation of the invention is now provided in connection with FIGS. 1-14.

FIG. 1 is a first perspective view of a laparoscopic device according to the invention. FIG. 1 is a partial view that shows the main shaft 24 of the device and the jaw assembly 25, which is comprised of an upper jaw 13 and a lower jaw 11. In this embodiment of the invention, the upper jaw is pivotable away from and toward the lower jaw about a pivot point 17, which, in this embodiment is comprised of a pin or axle. In other embodiments of the invention, the lower jaw may be pivotable as well, but in the embodiment shown in FIG. 1, the lower jaw is fixed. Pivoting of the upper jaw is accomplished by transmitting tension to a jaw activation pin 18, which is moveable, in an activation slot 19. Typically, tension is applied via a cable attached to the jaw activation pin. Thus, movement of the jaws is accomplished. The jaws themselves are configured for such laparoscopic procedures as electrocautery and tissue severing. Accordingly, as shown in the bottom jaw 11, a distal electrode 12 is provided, embedded in the plastic carrier 15. A second, proximal electrode 15 is also shown. A cutting groove 14 is shown for receiving a blade during a sectioning operation. The blade is not shown in FIG. 1.

During laparoscopic procedures, it is desirable to be able to position the jaws of the device from left to right to achieve the best angle of approach to the tissue to be treated. Key to the invention is the provision of an articulated wrist 22, which is comprised of a plurality of articulation discs or vertebrae 21. The articulation is accomplished by tensioning a pair of cables discussed below and a termination of which is shown in FIG. 1 as a wire, which is soldered or crimped in a groove at a cable termination point 20. Further, FIG. 1 shows a lock for an outside shaft tube or clamping mechanism to hold the wrist to the tube. This is shown by a clamping slot 23.

FIG. 2 is a top or plan view of the laparoscopic device showing the jaws 25 and shaft 24. In particular, the articulated Wrist 22 is shown in greater detail. In this embodiment a plurality of vertebrae comprises interconnected pivotable, hinged disks, where the discs 21 are articulated with one another and comprise a series of ball-like projections 27, which are engaged in complementary grooves 28. The jaw assembly 25 in this embodiment shows a ball-like projection 29, which is engaged in a groove of the articulation disc and the shaft 24 includes a complementary groove 30 for receiving a ball-like projection of an articulating disc. As can be seen in FIG. 2, a cable 31 is shown as well. The cable is a coiled pipe sheath assembly that, in this embodiment, is used to operate a blade within the jaw. The coiled assembly allows the cable to bend with the articulation of the device without kinking, as discussed above.

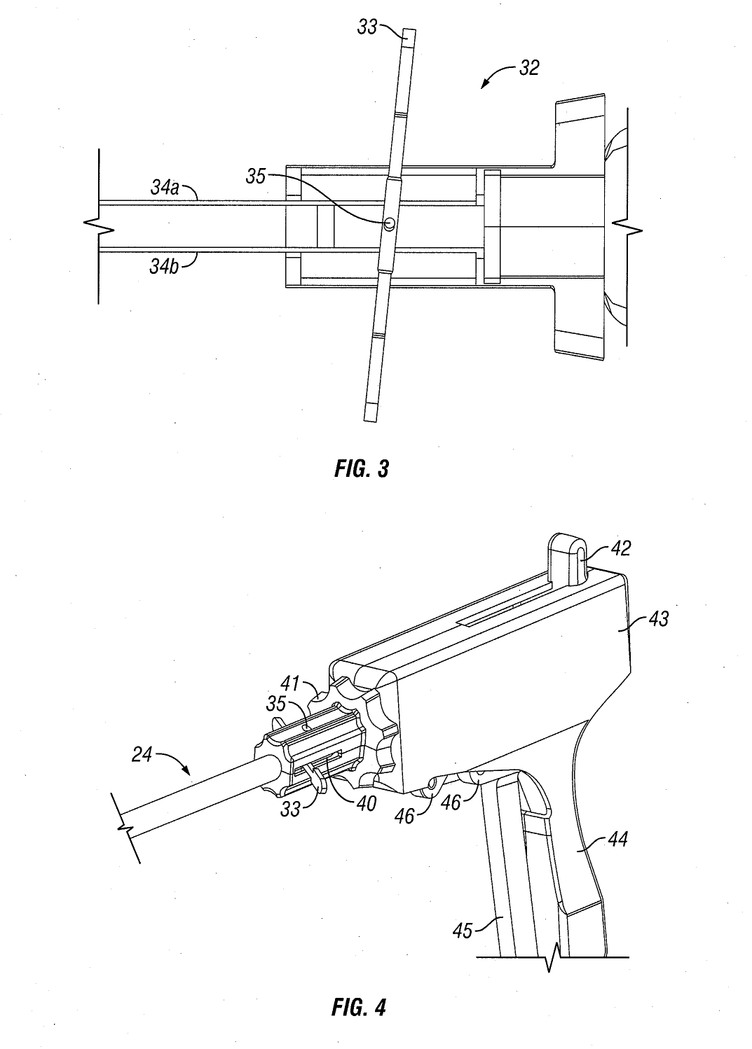

FIG. 3 is a partially cutaway side schematic view of an activation mechanism 32 for operating the articulation joint. In FIG. 3, a wrist articulation control 33 is shown having two finger-actuated blades to pivot the control about a pivot point 35. This pivoting action respectively applies attention to and draws attention from a pair of control cables 34a/34b, which, in this embodiment of the invention, are pretensioned Nitinol cables. Those skilled in the art will appreciate that other cable materials may be used. Operation of the wrist articulation control causes one cable to pull on the jaw assembly 25, thus causing the jaw assembly to move left or right as desired. Key to the invention is the provision of the articulation discs which allow such articulation to occur. In the prior art, it is known that such mechanisms as kerfs or other bending mechanisms can be provided. However, these mechanisms are subject both to stress, which reduces their effectiveness over time, and they retain a memory effect, such that there is a tendency for them to return to their initial position, rather the maintain a position desired by the surgeon using the device. The invention herein avoids both of these deleterious effects of operation of the device.

FIG. 4 is a perspective view of a laparoscopic device according to the invention showing a housing 43 having a handle 44 and a jaw activation trigger 45 that operates a four-bar linkage or other type of linkage 46 to transmit tension through the main shaft 24 and thereby operate the jaws to open and close them as desired. Also shown in FIG. 4 is a blade actuator 42 by which a blade may be drawn through the groove discussed above. A shaft rotator 41 allows the shaft to be rotated about a shaft access, while the wrist articulation control 33 allows the wrist mechanism to be operated. Note in FIG. 4 that the wrist articulation control includes a control slot 40 that both guides and contains the travel of the wrist articulation control 33.

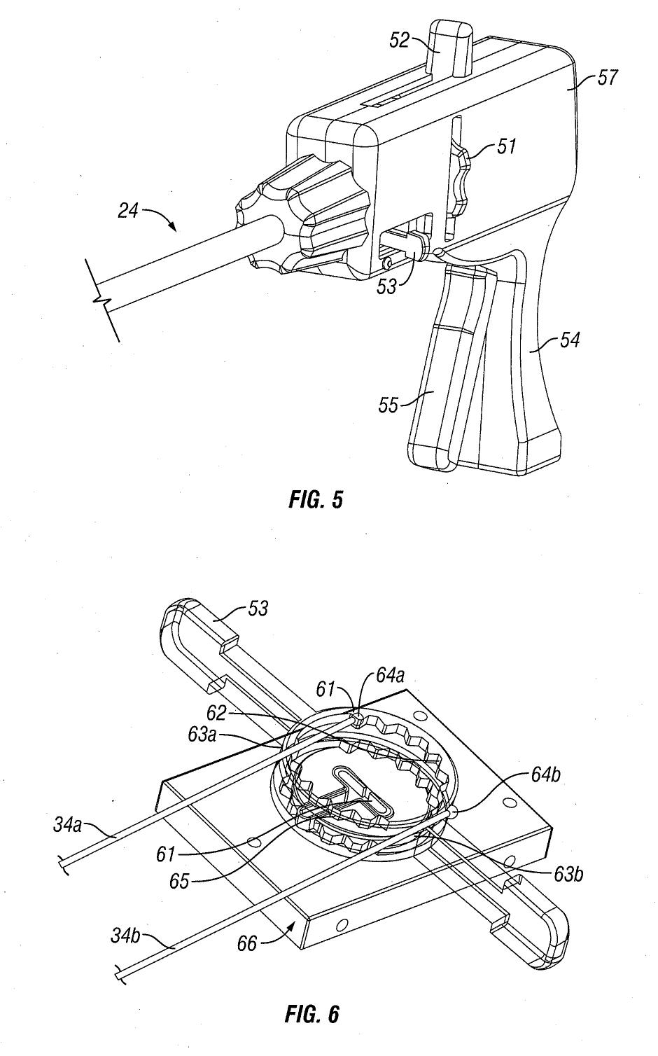

FIG. 5 is a perspective view of a further embodiment of the invention in which the shaft rotator 51 is contained within the housing 57. This embodiment of the invention also includes a blade actuator 52, a wrist articulation control 53, a handle 54, and a jaw activation trigger 55.

FIG. 6 is a perspective schematic view of the wrist activation control of the laparoscopic device shown in FIG. 5. A base portion 66 supports a ring projection 65 which, in turn, accommodates the control 53. Tensioned cables 34a/34b are shown having termination balls, which provide a cable stop 64a/64b. The cables are threaded through the control actuator 53 through respective grooves 63a/63b. An indexing disc 61 includes a plurality of detents 62. A flat spring 61 is arranged to engage within said detents to provide a stop mechanism to secure the jaws in a selected position by preventing movement of the articulation control 53, except when desired by a user of the device.

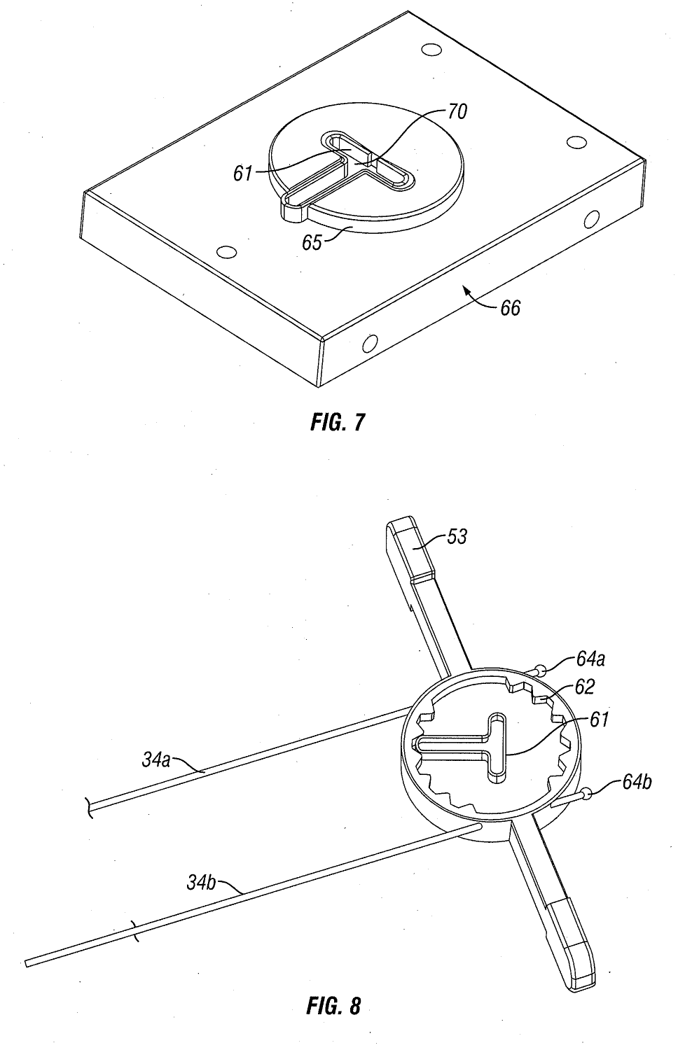

FIG. 7 is a perspective schematic view of the base portion of the articulation control 66 showing the spring mechanism 61 sitting in a recess 70 of the ring-like projection 65.

FIG. 8 is a schematic perspective view of the articulation control 53 showing the detents 62 in greater detail.

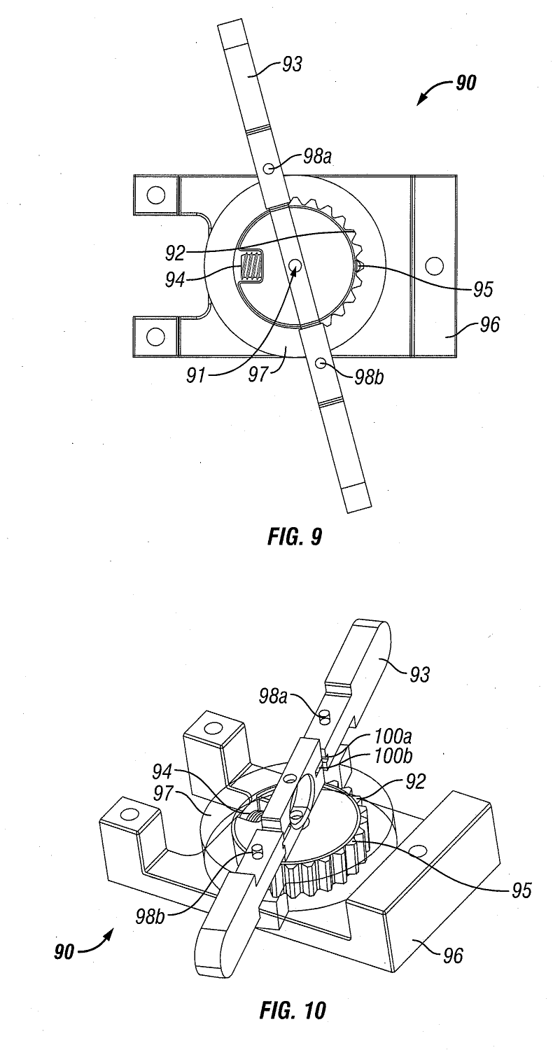

FIG. 9 shows an alternative embodiment invention, in which an articulation control 93 includes a plurality of detents 92 formed in a detent indexing disc 97. Operation of the control 93 causes rotation about a pivot point 91 and engages a step ball 95 into one of a plurality of detents 92 formed in the indexing ring. A ball plunger mechanism 94 maintains bias on the step ball 95. The indexing control 93 includes a pair of attachment points 98a/98b, discussed in greater detail below.

FIG. 10 is a perspective view of the index control mechanism for the articulation mechanism in the laparoscopic device. As shown in FIG. 10, a pair of grooves 100a/100b are provided for receiving control cables (not shown).

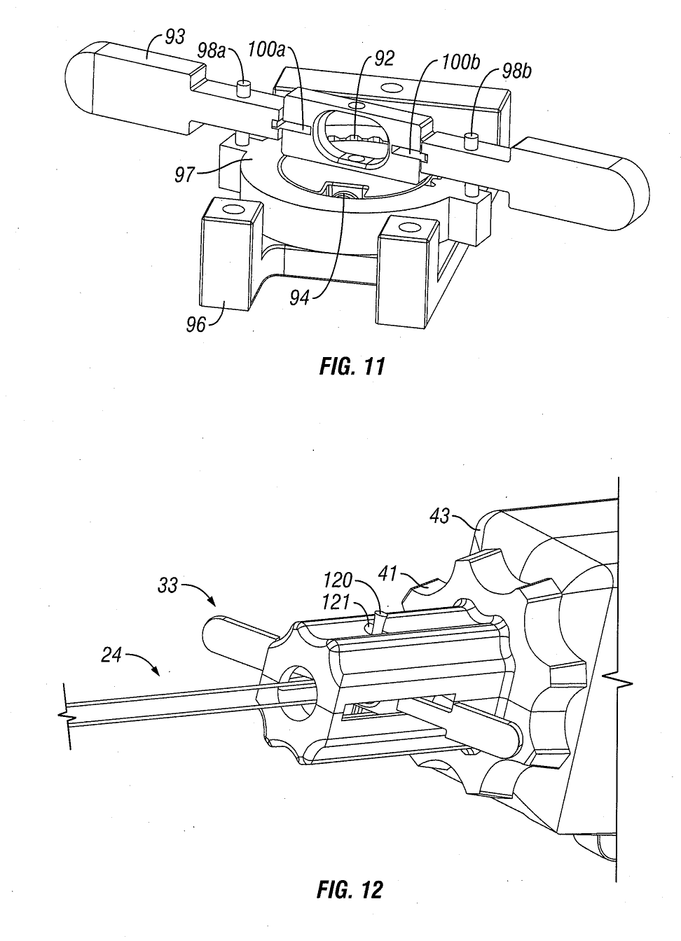

FIG. 11 is a further perspective view of the control mechanism for the articulation wrist in a laparoscopic device according to the invention. FIG. 11 shows clearly the arrangement of the articulation control 93 in connection with the indexing ring 97, and in particular shows the attachment there between a pair of pins 98a/98b.

FIG. 12 is a further embodiment of the invention showing an indexing mechanism comprised of an indexing pin 120, which is engaged in a slot 121.

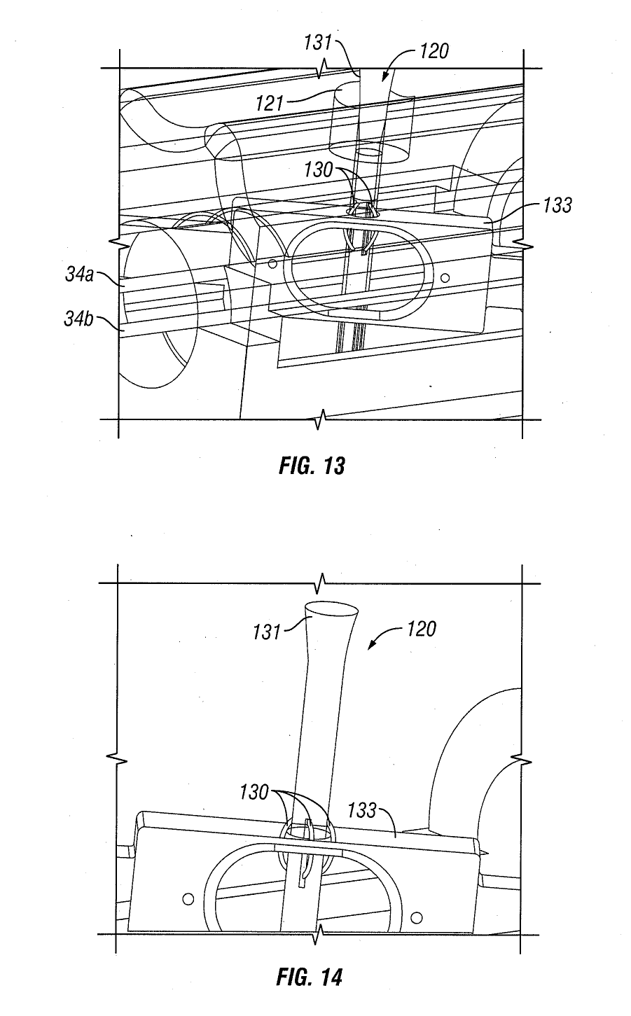

FIG. 13 is a cutaway perspective view showing the indexing pin 120 comprising a head portion 131 and a plurality of flared portions 130 which engage or disengage with a locking block 133. Accordingly, this embodiment of the invention comprises a jam lock in which depression of the pin 120 jams the flared portion of the pin 130 into the block 133 and thus prevents rotation of the actuation control mechanism.

FIG. 14 is a detailed view of the jam mechanism showing the pin 120, head 131, and flares 130 in greater detail.

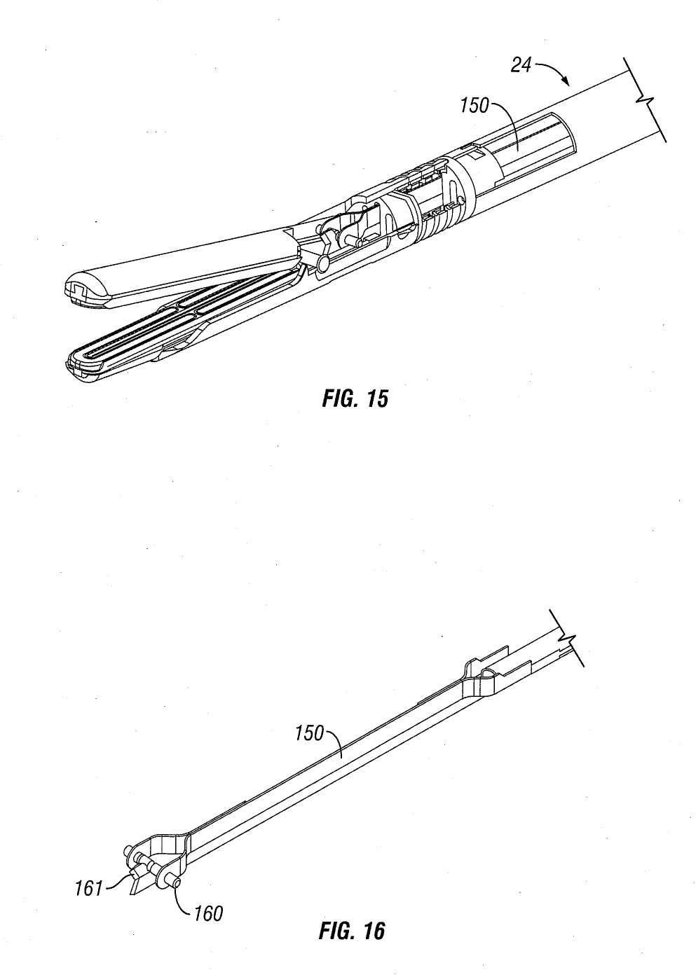

FIG. 15 is a perspective, partially cutaway view of a laparoscopic device, showing a drive member according to the invention. The drive members may be made of a round wire (stainless steel or Nitinol), using tightly wound coil springs for support. The drive members may also be flat stainless steel bands 150, as shown in FIGS. 15 and 16. FIG. 15 shows the wrist section of the device, while FIG. 16 shows only the components of interest, i.e., the jaw activating band 150, the closing pin 160, and cutting blade 161. This embodiment replaced the round wire with flat bands and supports the bands using the internal structure of the vertebrae. Other embodiments may use flat polymer bands to provide additional support. These bands could be either PTFE (Teflon®) or FEP. The support structure may also involve PTFE or FEP shrink tubing over the blade and/or the jaw actuation band.

Although the invention is described herein with reference to the preferred embodiment, one skilled in the art will readily appreciate that other applications may be substituted for those set forth herein without departing from the spirit and scope of the present invention. Accordingly, the invention should only be limited by the Claims included below.

1. A laparoscopic grasping instrument, comprising:
an elongated shaft having a set of opposing jaws associated with a distal end thereof and a handle associated with a proximal end thereof;
an articulation wrist positioned between said shaft and said jaw set for effecting movement of said jaws relative to said shaft as desired, both left and right, from centerline, said articulation wrist comprising:
an actuator associated with said handle;
at least one force transfer member for translating user operation of said actuator into movement of said jaws to control the degree of articulation of said instrument; and
an articulation wrist comprising a set of pivotal vertebra that are connected to each other, wherein each vertebra is adapted to pivot left and right from centerline in relation to any of said shaft and said jaw set.

2. The instrument of claim 1, further comprising:
a jaw actuation member.

3. The instrument of claim 1, further comprising:
a cutter; and
a cutter driving member.

4. The instrument of claim 1, further comprising:
a bendable support for said force transfer member comprising a tightly wound coil spring housed within said wrist joints to route said drive member.

5. The instrument of claim 1, further comprising:
a locking mechanism for preventing motion of said articulation wrist while said user performs other operations on said instrument.

6. The instrument of claim 5, said locking mechanism further comprising:
an indexing mechanism with which said user can index and choose a necessary amount of jaw angle between preset angles.

7. The instrument of claim 1, said force transfer member comprising:
one or more wires or cables that run down both sides of the articulation wrist for transferring forces from said actuator to said wrist.

8. The instrument of claim 1, said actuator comprising:
a rotating assembly that receives said one or more force transfer members from said wrist;
wherein said rotating assembly is mounted to pivot about a pivot point at said handle; and
wherein said actuator is configured for concentric rotation about said pivot point;
wherein length-wise motion of said one or more force transfer members along said shaft is controlled, based upon a distance from said pivot point to an attachment point of said one or more force transfer members.

9. The instrument of claim 8, wherein an angle of articulation is controlled by a distance that said one or more force transfer members move, which is predetermined by said wrist geometry.

10. The instrument of claim 5, said locking mechanism further comprising:
a spring having a shape that deflects when a force is applied thereto;
wherein said spring is housed within a circular carrier; and
wherein only the deflectable portion of said spring accessible and protruding from said circular carrier;
a rotating member having a circular portion removed from a pivot area;
wherein said rotating member is adapted to fit over said circular carrier; and
a tooth pattern that is removed from along an inner diameter of said circular portion of said rotating member;
said rotating member comprising arms extending from a center body thereof to which said one or more force transfer members are attached;
wherein said spring protrudes into indentations created by said tooth pattern; and
wherein an angle of articulation of said wrist is controlled by predetermining distances between said teeth and a distance from an attachment point of said one or more force transfer member to pivot point.

11. The instrument of claim 5, said locking mechanism further comprising:
a spring biased ball plunger mounted within a circular carrier;
a rotating member having a circular portion removed from a pivot area;
wherein said rotating member is adapted to fit over said circular carrier; and
a tooth pattern that is removed from along an inner diameter of said circular portion of said rotating member;
wherein said spring plunger mates with indents created by said tooth pattern.

12. The instrument of claim 5, said locking mechanism further comprising:
a wing is mounted on top of a rotating member;
wherein said wing is adapted to be manipulated to control rotation of said rotating member around a circular carrier.

13. The instrument of claim 5, said locking mechanism further comprising:
a living hinge mounted near said handle, said living hinge having a V-shape portion that is adapted to fit within a series of slots of an external housing that surrounds said living hinge, said V-shape portion having a tip that protrudes from said series of slots;
wherein said series of slots are disposed along a length of said external housing;
wherein said housing engages with said at least one force transfer member;
wherein said articulation wrist is adapted to be adjusted and locked by first pressing down on said living hinge to disengage a current locked position, and then moving said external housing from a proximal to a distal position or vice versa, which then locks by re-engaging with said living hinge at any various predetermined distances set by said series of slots, wherein said distances determine an angle at which said wrist is articulated.

14. The instrument of claim 5, said locking mechanism further comprising:
a rotating member that rotates freely about a pivot pin;
a wedge-like, tube-shaped button mounted on top of said pivot pin and anchored within said pivot pin, said button adapted to be depressed into said pivot pin, wherein a portion of said button flares through a slot formed in said pivot pin to lock said wrist angle and said rotating member at a user determined angle of articulation.

15. The instrument of claim 1, said jaw set further comprising:
at least one set of electrodes for receiving an electric charge and for imparting said charge to an organ or tissue during an electrocautery procedure.

16. The instrument of claim 1, each said vertebrae comprising:
a ball-like projection and a complementary groove;
wherein a ball-like projection of one vertebrae is adapted for engagement in a complementary groove of an adjacent vertebrae.

17. The instrument of claim 1, each said vertebrae comprising:
a pivotable, hinged disk.

18. The instrument of claim 1, further comprising an instrument drive member that effects selected instrument operation in response to selected user instrument activation.

19. The instrument of claim 18, said drive member comprising a round wire supported by tightly wound coil springs.

20. The instrument of claim 19, said round wire comprising either of stainless steel or Nitinol.

21. The instrument of claim 18, said drive member comprising a flat band.

22. The instrument of claim 18, said instrument operation comprising movement of a blade.
